(19) United States
(12) Patent Application Publication
(10) Pub. No.: US 2023/0399558 A1
(43) Pub. Date: Dec. 14, 2023

(54) HEAT TRANSFER LIQUID FOR A TEMPERATURE CONTROL DEVICE FOR EXTRACORPOREAL CIRCULATION

(71) Applicant: LivaNova Deutschland GmbH, Munich (DE)

(72) Inventors: Erwin Knott, Poing (DE); Thomas Heckmaier, Munich (DE); Filip De Somer, Ghent (DE)

(73) Assignee: LivaNova Deutschland GmbH, Munich (DE)

(21) Appl. No.: 18/239,408

(22) Filed: Aug. 29, 2023

Related U.S. Application Data
(63) Continuation of application No. 16/781,925, filed on Feb. 4, 2020, now abandoned.
Application No. 16/781,925 is a continuation of application No. 15/505,024, filed on Feb. 17, 2017, now abandoned.

(51) Int. Cl. C09K 5/10; A01N 59/00; A61L 2/18; A61M 1/36; A61F 7/00

(52) U.S. Cl. CPC C09K 5/10; A01N 59/00; A61L 2/186; A61M 1/367; A61M 1/3623; A61F 7/0085; A61L 2202/24; A61M 2205/366

(57) ABSTRACT

The present application refers to a heat transfer liquid for a temperature control device for human body temperature control during extracorporeal circulation, a temperature control device, the use of heat transfer liquid for extracorporeal circulation and a method of human body temperature control using a heat transfer liquid. The head transfer liquid consists of ethylene glycol or propylene glycol at about 25 volume-percent to about 35 volume-percent, hydrogen peroxide at 0.05 volume-percent or less and sterile, filtered and de-mineralized water as rest.

### CROSS-REFERENCE TO RELATED APPLICATION

This application is a continuation of U.S. patent application Ser. No. 16/781,925, filed Feb. 4, 2020, which is a continuation of U.S. patent application Ser. No. 15/505,024 filed Feb. 17, 2017, which is a national stage application of International Application No. PCT/EP2014/067746, filed Aug. 20, 2014, the contents of which are herein incorporated by reference in their entirety.

### TECHNICAL FIELD

The present disclosure relates to a heat transfer liquid for a temperature control device for human body temperature control during extracorporeal circulation, a temperature control device for human body temperature control and a method of human body temperature control during extracorporeal circulation.

### BACKGROUND

Extracorporeal circulation of blood is used in certain surgical procedures such as during heart surgery. During the extracorporeal circulation, the body temperature of the patient can be controlled, by controlling the temperature of the blood during extracorporeal circulation. For this purpose, a patient temperature control system can be provided by means of which the temperature of the blood of the patient in the circulation can be raised or lowered. The blood thus controlled, flows through the patient and the body of the patient approaches the temperature of the blood. So as to heat or cool the blood, the temperature control system may comprise a heater and/or cooler device providing a liquid circulation to a disposable (single use) heat exchanger that transfers energy to and/or away from the patient's blood circulation. Conventionally, the liquid is water.

The heat exchanger for the blood is a strict dual circuit system, the blood side and the liquid side being separated from each other so that any mixture, such as by means of diffusion, between the blood in one of the circuits and the temperature control liquid in the other of the circuits is inhibited as much as possible. Nevertheless, care has to be taken to avoid health risks stemming from the liquid.

### SUMMARY

The applicant has designed a mobile temperature control device for human body temperature control during extracorporeal circulation. Such a mobile device can be connected to a circuit of temperature control liquid to be used in a heat exchanger. The mobile temperature control device preferably is provided with exchangeable hoses or tubes or other conduits. Further, connecting and disconnecting these conduits can more easily be achieved if the conduits are not filled with the temperature control liquid during connecting and disconnecting. Likewise, it is preferred if the circuit can be emptied of temperature control liquid for the connection and disconnection of the mobile temperature control device. The preferred mobile device is consequently provided with an open reservoir where the temperature control liquid is exposed to environmental air. The temperature control liquid can be fed into the circuit from this reservoir and can be returned to it. Any air which might be trapped in one of the conduits during connecting or disconnecting the conduit and the temperature control device or the heat exchanger can be bled to the environment via the open reservoir. This means, however, that the temperature control liquid is exposed to the air of the environment.

Substances used as temperature control liquid, in particular water, are prone to microbial contamination when exposed to environmental air. If the temperature control liquid was exposed to the environmental air, disinfecting the temperature control device and the temperature control liquid can then improve the microbial status of the liquid and render the mobile device maintenance- and service-friendly.

Usually, the microbial growth in water is rapid. The microbial population in water of drinking quality, which has a microbial population of less than 100 cfu/ml, grows within 14 days to as much as 1,000,000 cfu/ml. Therefore, it was essential for a sterile operation of extracorporeal circulation that the water used as the heat transfer liquid for human body temperature control during extracorporeal circulation was exchanged regularly.

Further, in the prior art, disinfecting the temperature control device, i.e. the heater and/or cooler, was necessary in view of the rapid growth of the microbial population in water. It was, for example, accomplished by regularly disassembling all devices and subjecting the disassembled parts to a separate disinfection procedure. This was both time consuming and expensive. Further, it was required to establish a monitoring and recordation system in order to maintain and verify the disinfection status of the heater and/or cooler system within an acceptable range.

In view of the above situation, it is presently required to spend a lot of time, effort and money on the maintenance of a temperature control device for human body temperature control during extracorporeal circulation.

The present disclosure addresses the problem of the high amount of time, effort and. money to be spent in order to avoid a hygienic risk for the patient and the sterile environment in an operating room in which extracorporeal circulation is carried out.

This problem is solved by the inventive heat transfer liquid according to claim 1, the temperature control device according to claim 4, the use of the heat transfer liquid according to claim 5 and the method of human body temperature control according to claim 6. Further advantageous features and embodiments are defined in the dependent claims.

According to the present disclosure, the heat transfer liquid for a temperature control device for human body temperature control during extracorporeal circulation consists of ethylene glycol or propylene glycol at about 25 volume-percent to about 35 volume-percent, hydrogen peroxide at 0.05 volume-percent or less, preferably about 0.01 volume-percent to about 0.03 volume-percent, and sterile, filtered and demineralized water as rest.

On the basis of a heat transfer liquid consisting of between about 25 volume-percent and about 35 volume-percent of ethylene glycol or propylene glycol and water at between 65 volume-percent and 75 volume-percent, the inventors have found that a very small amount of hydrogen peroxide can be used as a long-term disinfectant which renders regular disinfecting the device or exchanging of the heat transfer liquid unnecessary. Moreover, since the heat transfer liquid for a temperature control device for human body temperature control during extracorporeal circulation may, e. g. due to a leaking device, contact or mix with the blood of the patient, it is of ultimate importance that the heat transfer liquid is non-toxic and has no detrimental effects on the health status of the patient.

Both propylene glycol and hydrogen peroxide carry the designation of the U.S. Food and Drug Administration (FDA) of “Generally Recognized As Safe” (GRAS). Both substances are also used as food additives. These substances, at least in a food grade concentration, are safe also as a disinfectant in a heat transfer liquid for extracorporeal circulation, as the inventors have found.

According to the disclosure, microbial contamination and, as a consequence, the need for regular disinfection and replacement of the heat transfer fluid can be avoided by using the heat transfer liquid according to the present disclosure. This significantly reduces the maintenance efforts for a temperature control device for human body temperature control during extracorporeal circulation and even increases the safety for the patient during extracorporeal circulation since microbial contamination is prevented at all times.

For water to be considered as sterile, filtered and demineralized water in accordance with the present disclosure, the initial water contamination is close to 0 cfu. This result is achieved by filtering normal tap water through a water filter having a filter mesh size of 0.2μαl. As normal bacteria and algae are sized i πi or larger, these microorganisms are not able to pass the filter, resulting in sterile or close to sterile water after the filter process.

Preferably, the heat transfer liquid comprises ethylene glycol or propylene glycol at about 30 volume-percent. This preferred amount of ethylene glycol or propylene glycol in the heat transfer liquid allows for an optimum liquid with particular reference to its bacteriostatic properties.

It is further preferred that the hydrogen peroxide is comprised in the heat transfer liquid at about 0.02 volume-percent. This concentration of hydrogen peroxide in the heat transfer liquid ensures an effective bactericide effect while maintaining the amount of hydrogen peroxide in the heat transfer liquid at a low level.

### BRIEF DESCRIPTION OF THE DRAWINGS

FIG. 1 shows a schematic diagram illustrating a temperature control device in a heat exchanging system.

### DETAILED DESCRIPTION OF PREFERRED EMBODIMENT

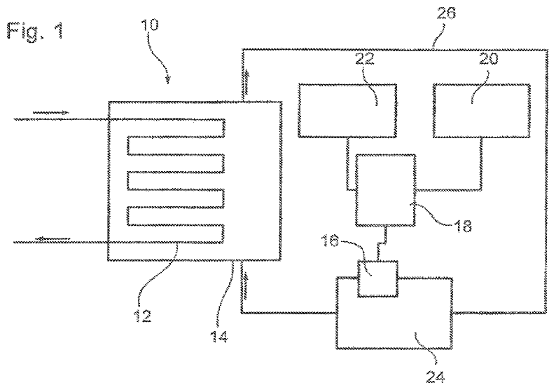

FIG. 1 shows a schematic diagram of a heat exchanger 10 for human body temperature control during extracorporeal circulation. The heat exchanger 10 comprises a blood side 12 and a heat transfer liquid side 14 in which blood and a heat transfer liquid are respectively circulated.

The temperature of the heat transfer liquid used in the heat exchanger 10 is controlled by a heater and/or cooler 24, i.e. a temperature control device. The heater and/or cooler 24 is a device which is capable of heating or cooling or heating and cooling the heat transfer liquid. Preferably, the heater or cooler is capable of both heating and cooling the heat transfer liquid so that a defined temperature of the heat transfer liquid can be maintained. The heater and/or cooler 24 is part of a heat transfer liquid side circuit 26 in which the heat transfer liquid is circulated for operating the heat exchanger 10 in controlling the temperature of the human body of a patient.

The heater and/or cooler 24 may be provided with a disinfectant sensor 16 configured for measuring a concentration of a disinfectant in the heat transfer liquid in the heater and/or cooler 2. The result of the measurement of the sensor 16 may be transmitted to a computer 18 which may control a first display 20 which may indicate the measured concentration of disinfectant in the heat transfer liquid and a second display 22 which may indicate the consequence of the measured concentration. In particular, the second display 22 may indicate whether the disinfection status of the heat transfer liquid is ok, whether disinfectant is to be added or whether the heat transfer liquid is to be exchanged.

The heat transfer liquid circulated in the heat transfer liquid side circuit 26 of the present embodiment consists of ethylene glycol or propylene glycol at 30 volume-percent, hydrogen peroxide at 0.02 volume-percent and sterile, filtered and de-mineralized water as rest. In particular the hydrogen peroxide may be considered by the sensor 16 as disinfectant and a concentration of hydrogen peroxide may be measured by the sensor 16. But also a concentration of ethylene glycol or propylene glycol can be determined by the sensor 16 or a different sensor.

Measuring the concentration of disinfectant, be it hydrogen peroxide, ethylene glycol or propylene glycol is, however, optional and not essential for the present disclosure.

What is claimed is:

1. A temperature control system for human body temperature control during extracorporeal circulation, comprising:
a temperature control liquid including sterile, filtered and demineralized water, and a disinfectant to inhibit microbial growth;
a temperature control device adapted to heat or cool the temperature control liquid;
at least one disinfectant sensor for measuring a concentration of the disinfectant in the temperature control liquid;
a heat exchanger having a blood flow portion and a temperature control liquid flow portion, the heat exchanger configured to alter the temperature of blood flowing through the blood flow portion; and
a computer coupled to the disinfectant sensor, the computer configured to indicate a measured concentration of disinfectant in the temperature control liquid, and to indicate a disinfection status of the temperature control liquid;
wherein the temperature control system is adapted to heat or cool the heat transfer liquid based on an input provided by a user to the computer.

2. The temperature control system of claim 1, further comprising first and second displays, the computer configured to control the first display to display the measured concentration of disinfectant and to control the second display to indicate the disinfection status.

3. The temperature control system of claim 1, wherein the disinfection status includes a recommendation to add more disinfectant.

4. The temperature control system of claim 1, wherein the disinfection status includes a recommendation to exchange the temperature control liquid.

5. The temperature control system of claim 1, wherein the heat exchanger includes an open reservoir where the temperature control liquid is exposed to environmental air.

6. The temperature control system of claim 5, further comprising exchangeable conduits configured to be coupled to the open reservoir.

7. The temperature control system of claim 1, wherein the disinfectant includes at least 25 volume percent of ethylene glycol or propylene glycol.

8. The temperature control system of claim 7, wherein the disinfectant includes no more than 35 volume percent of ethylene glycol or propylene glycol.

9. The temperature control system of claim 7, wherein the disinfectant includes hydrogen peroxide.

10. The temperature control system of claim 9, wherein the disinfectant includes to 0.03 volume percent hydrogen peroxide.

11. The temperature control system of claim 9, wherein the disinfectant includes 30 volume percent of ethylene glycol or propylene glycol, and 0.02 volume percent hydrogen peroxide.

12. The temperature control system of claim 1 wherein the heat exchanger has a first inlet to receive the heat transfer liquid and a first outlet to remove heat transfer liquid and further wherein the heat exchanger has a second inlet to receive blood from the human body and a second outlet to remove the blood from the heat exchanger.

13. A temperature control system for human body temperature control during extracorporeal circulation, comprising:
a temperature control liquid including sterile, filtered and demineralized water, and a disinfectant to inhibit microbial growth, wherein the disinfectant includes both hydrogen peroxide and ethylene glycol or propylene glycol;
a temperature control device adapted to heat or cool the temperature control liquid;
at least one disinfectant sensor for measuring a concentration of the disinfectant in the temperature control liquid;
a heat exchanger having a blood flow portion and a temperature control liquid flow portion, the heat exchanger configured to alter the temperature of blood flowing through the blood flow portion; and
a computer coupled to the disinfectant sensor, the computer configured to indicate a measured concentration of disinfectant in the temperature control liquid, and to indicate a disinfection status of the temperature control liquid, wherein the disinfection status includes a recommendation to add more disinfectant or to exchange the temperature control liquid;
wherein the temperature control system is adapted to heat or cool the heat transfer liquid based on an input provided by a user to the computer.

14. The temperature control system of claim 13, further comprising first and second displays, the computer configured to control the first display to display the measured concentration of disinfectant and to control the second display to indicate the disinfection status.

15. The temperature control system of claim 13, wherein the heat exchanger includes an open reservoir where the temperature control liquid is exposed to environmental air.

16. The temperature control system of claim 15, further comprising exchangeable conduits configured to be coupled to the open reservoir.

17. The temperature control system of claim 13, wherein the ethylene glycol or propylene glycol is present in a concentration of at least 25 volume percent.

18. The temperature control system of claim 17, wherein the ethylene glycol or propylene glycol is present in a concentration of no more than 35 volume percent.

19. The temperature control system of claim 17, wherein the hydrogen peroxide is present in a concentration of 0.01 to 0.03 volume percent.

20. The temperature control system of claim 19, wherein the ethylene glycol or propylene glycol is present in a concentration of 30 volume percent, and the hydrogen peroxide is present in a concentration of 0.02 volume percent.
